(19) United States
(12) Patent Application Publication
(10) Pub. No.: US 2010/0286629 A1
(43) Pub. Date: Nov. 11, 2010

(54) Port

(76) Inventor: Walter Egle, Koblach (AT)

Correspondence Address: WENDEROTH, LIND & PONACK, L.L.P., 1030 15th Street, N.W.,, Suite 400 East, Washington, DC, 20005-1503, US

(21) Appl. No.: 12/078,726

(22) Filed: Apr. 3, 2008

(30) Foreign Application Priority Data
Mar. 14, 2008 (AT) A 410/2008

(51) Int. Cl. A61M 37/00

(52) U.S. Cl. 604/288.02

(57) ABSTRACT

A port for implantation under the skin of a human or animal body comprises a housing (1) within which an interior chamber (2) is located and which has an opening (4) closed by a diaphragm (3) which, for the purpose of introducing a fluid into the interior chamber (2), can be pierced by an injection needle. The housing (1) includes at least one recess (13) spaced apart from the opening (4), which recess encompasses at least to a large extent the opening (4), or several recesses (13′) spaced apart from the opening (4), which together encompass at least to a large extent the opening (4).

### BACKGROUND OF THE INVENTION

a) Field of the Invention

The invention relates to a port for implantation under the skin of a human or animal body, with a housing, within which an interior chamber is located and which includes an opening closed by a diaphragm which can be pierced by an injection needle for the introduction of a fluid into the interior chamber.

b) Description of Related Prior Art

Such ports are utilized for port catheters for the introduction of a fluid, for example for drug therapy, into a hollow organ of a human or animal body. For example, in chemotherapy, drugs are introduced in this way directly into the blood circulation. Apart from the introduction of fluids into blood vessels, fluids can also be introduced with port catheters directly into the gastrointestinal tract, the bladder and other hollow organs.

Ports of the above described type are further employed as injection ports for the introduction of a fluid into a band finable with a fluid, which band serves for the placement about a hollow organ of a human or animal body. Such bands are, for example, utilized as gastric bands for the treatment of obesity.

Such conventional ports entail the problem that the treating physician, who wishes to inject fluid into the port, can miss the opening in the housing through which the diaphragm is accessible, whereupon the tip of the needle can slide along the top side of the housing and can penetrate into the subjacent tissue. The risk entailed therein is damaging potentially sensitive tissue structures.

### PROBLEM ADDRESSED BY THE INVENTION AND SUMMARY OF THE INVENTION

The invention addresses the problem of providing a port of the above described type, through which the patient safety is improved. This is attained according to the invention through a port for implantation under the skin of a human or animal body, with a housing, within which an interior chamber is located and which includes an opening closed by a diaphragm, which diaphragm can be pierced by an injection needle for the purpose of introducing a fluid into the interior chamber, the housing comprising at least one recess spaced apart from the opening, which, at least to a large extent, encompasses the opening, or several recesses spaced apart from the opening, which together, at least to a large extent, encompass the opening.

The at least one recess which encompasses at least to a large extent the opening, or one particular one of the several recesses, which together at least to a large extent encompass the opening, can receive the tip of an injection needle. Thus, when piercing the injection needle through the skin of the patient, the opening is missed, the injection needle sliding off along the top side of the housing can be caught with its tip in the recess, or in one of the recesses, such that the injection needle does not slide entirely off the housing and damage the subjacent tissue.

The at least one recess encompassing at least to a large extent the opening of the housing, which opening is disposed in a roof wall of the housing, or the recesses, which together at least to a large extent encompass the opening disposed in the roof wall of the housing, is or are preferably located in the top third of the height of the housing. This top third of the height of the housing adjoins a top edge of the roof wall of the housing. Thereby a possible sliding off of the injection needle along the top side of the housing can already be intercepted relatively close to the surface of the skin. The risk that the sliding needle can pass the at least one recess is thereby decreased. Punctures are also more painful and more harmful to the patient, the deeper they penetrate.

In an advantageous embodiment the port with a tube connected to it forms a port catheter for the introduction of a fluid into a hollow organ of a human or animal body.

In another embodiment a port according to the invention can form an injection port for the introduction of a fluid into a hollow chamber of a band, which band serves for the implantation around a hollow organ, for example the stomach, of the human or animal body.

Further advantages and details of the invention will be explained in the following in conjunction with the enclosed drawing.

### BRIEF DESCRIPTION OF THE DRAWING

In the drawing depict:

FIG. 1 an oblique view of a port with a section of a connected tube according to a first embodiment of the invention in oblique view [sic],

FIG. 2 an oblique view from a different viewing direction,

FIG. 3 a top view,

FIG. 4 a section along line AA of FIG. 3,

FIG. 5 a top view onto a further feasible embodiment of the invention.

The Figures are drawn to different scales.

### DESCRIPTION OF THE PREFERRED EMBODIMENT EXAMPLES

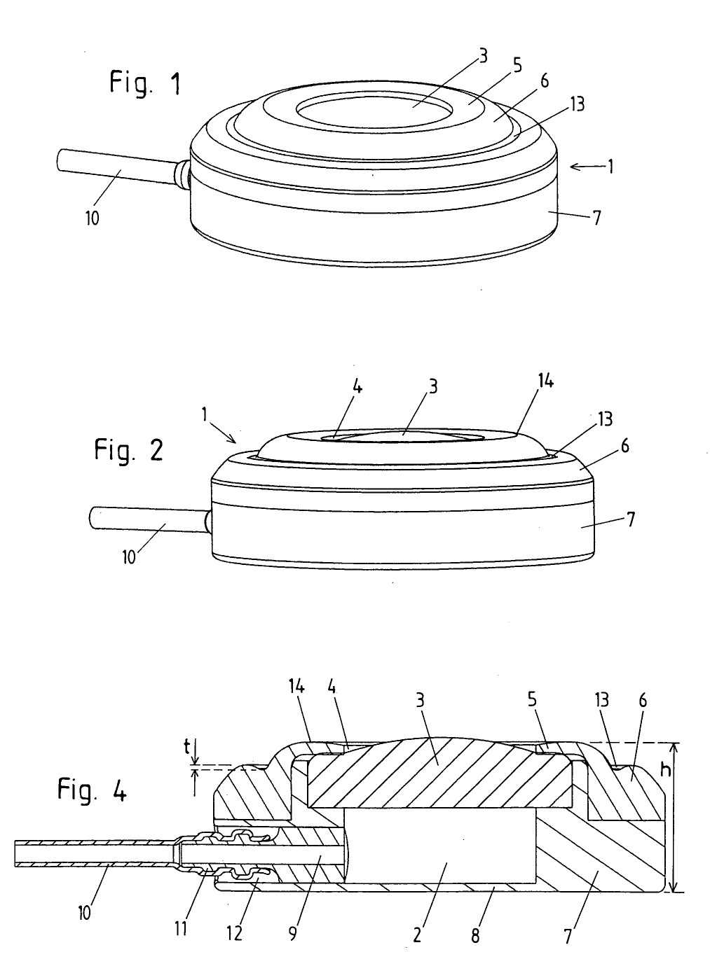

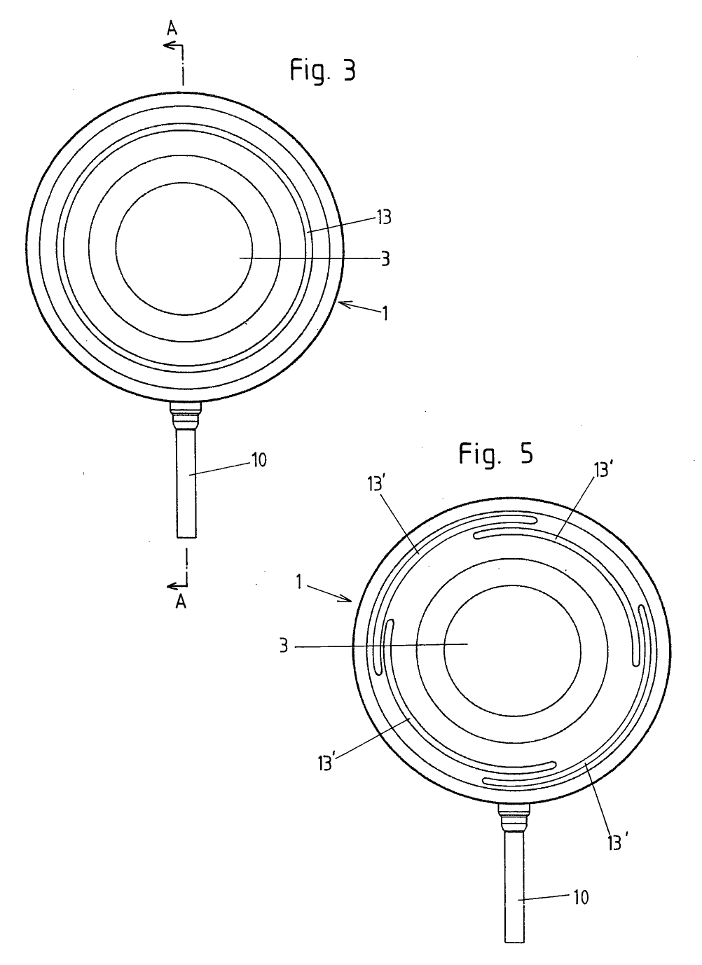

An embodiment example of a port according to the invention is depicted in FIGS. 1 to 4. The port comprises a housing 1 within which an interior chamber 2 is located and which includes an opening 4 located in a roof wall 5, the opening 4 being closed by a diaphragm 3.

In order to introduce a fluid into the chamber 2, the diaphragm 3, comprised in particular of silicon, is pierced in the region of the opening 4 with an injection needle and the fluid is injected into the chamber 2. After the injection needle is withdrawn, the puncture opening of diaphragm 3, as is known, closes due to the elasticity of the material of diaphragm 3.

In the depicted embodiment example, the housing 1 comprises a top housing part 6, which includes the roof wall 5 with opening 4, and a bottom housing part 7, which includes a bottom wall 8. The top housing part 6 and the bottom housing part 7 can be comprised, for example of metal, for example titanium.

The top housing part 6 can be pressed onto the bottom housing part 7 to connect them with one another. The top housing part 6 could, for example, also be connected with the bottom housing part 7 by adhesion or screw connection. To seal the chamber 2, a seal of an elastic material could also be provided between the top housing part 6 and the bottom housing part 7.

The housing 1 can also be formed of more than two housing parts.

From the chamber 2 of housing 1 leads a channel 9 through the housing 1, via which channel fluid introduced into the chamber 2 can exit from it and enter a tube 10 connected to the housing 1. A section of such, in particular flexible, tube 10 connected to housing 1 is depicted in the Figures.

The channel 9 in the depicted embodiment example is developed by a connecting piece 11 to which the tube 10 can be connected. The connecting piece 11 is set, for example pressed or adhered, into a bore 12 in the bottom housing part 7, The channel 9 could, for example, also extend directly through one of the housing parts 6, 7. A connecting piece for a tube could herein also be formed integrally with one of the housing parts 6, 7.

In the depicted embodiment example the diaphragm 3 is inserted between the bottom housing part 7 and the top housing part 6, wherein it rests in contact on a step of the bottom housing part 7 and is pressed in between the bottom housing part 7 and top housing part 6 (FIG. 4 shows the diaphragm 3 in the relaxed state and therefore slightly overlapping with the roof wall 5 of the top housing part 6). The diaphragm 3 could, for example, also rest in contact on the top side (implemented flat) of the bottom housing part 7 or within the bottom housing part 7 an insert could be disposed on which the diaphragm 3 rests. The diaphragm 3 could also be, for example, adhered on the top housing part 6.

The height h measured between the bottom edge of bottom housing part 7 and top edge of top housing part 6 of housing 1 is indicated in FIG. 4. This height h is measured at right angles to the main plane of diaphragm 3.

In the depicted embodiment example in which the underside of the bottom wall 8 is formed, as preferred, flat, the height h is measured at right angles to the underside of bottom wall 8.

In a region surrounding the opening 4, the topside of housing 1 is provided with a groove-shaped recess 13, which annularly encompasses the opening 4. The direction in which the recess 13 opens and the axis of the opening 4 form an angle of less than 90°, preferably less than 45°. In the depicted embodiment they are equidirectional.

If a person treating the patient, into whom the port is subcutaneously implanted, misses the opening 4 when piercing the skin with an injection needle and if, as a consequence, the tip of the injection needle slides off along the housing (in a direction away from opening 4), the tip of the needle reaches the recess 13 and can be prevented by it from sliding further. The recess 13 has for this purpose sufficient width and depth.

The depth t of recess 13 is preferably at least 0.5 mm. The width of recess 13, measured at one half depth t, is preferably at least 1 mm.

A further feasible embodiment of the invention is shown in FIG. 5. It differs from the previously described embodiment example therein that here, instead of a single recess 13 annularly, in particular circularly, encompassing the opening 4, the topside of housing 1 is provided with several recesses 13′ which together encompass the opening 4 completely. Each of the recesses 13′ extends in the shape of a partial ring, offset with respect to one another, about opening 4, and recesses 13′ successive in the circumferential direction overlap in end sections.

For the preferred depths and widths of the recesses 13′ apply the statements made already in connection with recess 13 described in the first embodiment example.

Recess 13 depicted in the embodiment example of FIGS. 1 to 4, as the recess 13′ depicted in the embodiment example of FIG. 5, is also in the top third of the height h of housing 1. This top third of height h of the housing is that third of height h of the housing which adjoins the top edge 14 of the top housing part 6.

Recess 13 or recesses 13′ is or are formed by a contour of housing 1 which is curved without sharp edges in order to avoid damaging the skin of the patient.

The at least one recess 13 or the recesses 13′ is or are located in the depicted embodiment example in the top housing part 6. If the housing 1 is formed with a bottom housing part 7 extending sufficiently far in the upward direction, the at least one recess 13 could also be located in the bottom housing part 7 or the recesses 13′ could also be located in the bottom housing part 7. The disposition between two housing parts, for example between top housing part and bottom housing part, is conceivable and feasible.

Various modifications of the depicted embodiment example are conceivable and feasible without leaving the scope of the invention. For example, more or fewer than four recesses 13′ each [encompassing] the opening 4 in the form of partial rings, could also be provided. For example, two or more concentric recesses 13 each annularly encompassing the opening 4 could also be provided.

Recess 13, or recesses 13′ together, could also not completely encompass the opening 4 but only at least to a large extent, i.e. over more than 50% of its circumference, preferably over at least more than 80% of its circumference, in order to prevent an injection needle from sliding off at least over the partial ring-shaped region over which the recess 13 extends, or the recesses 13′ together extend.

As is evident based on the above description, the scope of the invention is not limited to the depicted embodiment examples but rather should be determined with reference to the attached claims together with their full range of feasible equivalents. While the preceding description and the drawing represent the invention, it is obvious to a person of skill in the art that various modifications can be carried out therein without leaving the true spirit and scope of the invention.

### LEGEND OF THE REFERENCE NUMBERs

1 Housing
2 Chamber
3 Diaphragm
4 Opening
5 Roof wall
6 Top housing part
7 Bottom housing part
8 Bottom wall
9 Channel
10 Tube
11 Connecting piece
12 Bore
13, 13′ Recess
14 Top edge

1. Port for implantation under the skin of a human or animal body, with a housing (1), within which an interior chamber (2) is located and which includes an opening (4) closed by a diaphragm (3) which can be pieced by an injection needle for the purpose of introducing a fluid into the interior chamber (2), the housing (1) comprising at least one recess (13), spaced apart from the opening (4), which recess at least to a large extent encompasses the opening (4), or several recesses (13′), spaced apart from the opening (4), which together encompass at least to a large extent the opening (4).

2. Port as claimed in claim 1, wherein the at least one recess (13) encompassing at least to a large extent the opening (4) disposed in a roof wall (5) of the housing (1), or the recesses (13′) which together encompass at least to a large extent the opening (4) disposed in the roof wall (5) of the housing (1), is or are located in the top third, extending up to a top edge (14) of the roof wall (5) of the housing (1), of height (h) of the housing (1).

3. Port as claimed in claim 1, wherein the at least one recess (13) encompassing at least to a large extent the opening (4) or the recesses (13′) together encompassing at least to a large extent the opening (4) is or are formed in the shape of grooves.

4. Port as claimed in claim 1, wherein the at least one recess (13) encompassing at least to a large extent the opening (4) or the recesses (13′) together encompassing at least to a large extent the opening (4) extends or extend annularly or in the form of a partial ring.

5. Port as claimed in claim 4, wherein the at least one recess (13) encompassing at least to a large extent the opening (4) or the recesses (13′) together encompassing at least to a large extent the opening (4) extends or extend circularly or in the form of a circular arc.

6. Port as claimed in claim 1, wherein in the case of several recesses (13′), which together encompass at least to a large extent the opening (4), each of two recesses (13′) successive in the circumferential direction have end sections overlapping in the circumferential direction.

7. Port as claimed in claim 1, wherein from the interior chamber (2) of the housing (1) leaves a channel (9) extending through the housing (1) for the connection of the chamber (2) of the housing (1) with a tube (10) connected to the housing (1).

8. Port as claimed in claim 1, wherein the housing (1) comprises a bottom housing part (7) and a top housing part (6) and the opening (4) is disposed in the top housing part (6).

9. Port as claimed in claim 8, wherein the at least one recess (13) encompassing at least to a large extent the opening (4) or the recesses (13′) together encompassing at least to a large extent the opening (4) is or are disposed in the top housing part (6).

10. Port as claimed in claim 1, wherein the at least one recess (13) encompasses the opening (4) completely or the recesses (13′) together encompass the opening (4) completely.

11. Port as claimed in claim 1, wherein the port together with a tube (10) connected to it forms a port catheter for the introduction of a fluid into a hollow organ of a human or animal body.

12. Port as claimed in claim 1, wherein the direction into which the at least one recess (13) opens or into which a particular one of the recesses (13′) opens, forms with the axis of the opening (4), closed by the diaphragm (3), of the housing (1) an angle of less than 90°.

13. Port as claimed in claim 12, wherein the angle is less than 45°.
